(19) United States
(12) Patent Application Publication
(10) Pub. No.: US 2024/0027331 A1
(43) Pub. Date: Jan. 25, 2024

(54) HAND-HELD SCANNING PROBE AND OPTICAL SCANNING SYSTEM

(71) Applicant: OPXION TECHNOLOGY INC., New Taipei City (TW)

(72) Inventors: MENG-TSAN TSAI, New Taipei City (TW); FENG-YU CHANG, New Taipei City (TW); BO-HUEI HUANG, New Taipei City (TW)

(21) Appl. No.: 17/938,968

(22) Filed: Sep. 7, 2022

(30) Foreign Application Priority Data
Jul. 25, 2022 (TW) 111127751

(51) Int. Cl. G01N 21/31

(52) U.S. Cl. CPC G01N 21/31; G01N 2201/0221; G01N 2201/08; G01N 2201/0636; G01N 2201/0633

(57) ABSTRACT

A hand-held scanning probe is included in an optical scanning system. The hand-held scanning probe includes a housing and an optical component. The optical component includes a first lens, a reflector, a two-dimensional beam scanning mechanism, a splitter and a second lens. The first lens is used to receive a laser beam split by a fiber-coupled splitter and convert the laser beam into a form of collimated light. The reflector is used to refract the laser beam. The two-dimensional beam scanning mechanism provides the laser beam to a surface for two-dimensional scanning, producing a swing beam. The splitter is used to separate a scanning end beam returned from the test specimen from an illumination beam into two different light paths. The second lens is used to focus the swing beam at the test surface to form the scanning end beam for scanning. An optical scanning system is also provided.

### CROSS-REFERENCE TO RELATED APPLICATION

This non-provisional application claims priority under 35 U.S.C. § 119(a) on Patent Application No(s). 111127751 filed in Taiwan, R.O.C. on Jul. 25, 2022, the entire contents of which are hereby incorporated by reference.

### BACKGROUND OF THE INVENTION

### 1. Field of the Invention

The present disclosure provides an optical coherence tomography, and in particular to a hand-held scanning probe and an optical scanning system.

### 2. Description of the Related Art

Optical coherence tomography (OCT) can be applied to scan a surface of an object. OCT technology is similar to ultrasonic technology, the main difference is that OCT is the use of near-infrared beam, after light passes through a test object, a backscattered signal will be produced through the structure of the test object, and then the depth structure information of the test object can be obtained by receiving the backscattered signals generated by different depth structures.

### BRIEF SUMMARY OF THE INVENTION

The inventor found that the hand-held scanning probe used in the traditional optical coherence tomography system is bulky and heavy, because the optical path of the optical structure in the hand-held scanning probe is designed to be vertical, that is, the direction of the OCT sample end beam incident to the test structure is perpendicular to the direction of light emitted by the hand-held scanning probe. In addition, the hand-held scanning probe has a significant crooked appearance in response to the above design and is not easy to hold.

In view of the shortcomings of the prior art, the inventor exhausted his mind to propose a hand-held scanning probe and an optical scanning system to make the hand-held scanning probe have the advantages of light weight, small size and easy to hold.

To achieve the above objective and other objectives, an aspect of the present disclosure provides a hand-held scanning probe, which is included in an optical scanning system. The hand-held scanning probe includes a housing and an optical component disposed in the housing. The optical component includes a first lens, a reflector, a two-dimensional beam scanning mechanism, a splitter and a second lens. The first lens is used to receive a laser beam that is split by a fiber-coupled splitter and convert the laser beam into a form of collimated light. The reflector is set relative to the first lens and has a first mirror surface. The first mirror surface is used to refract the laser beam to change the direction of the laser beam. The two-dimensional beam scanning mechanism has a second mirror surface. The second mirror surface is set relative to the first mirror surface to change the direction of the laser beam again and provide a swing beam to a test surface of a test specimen for two-dimensional mobile scanning. The splitter is set relative to the two-dimensional beam scanning mechanism, and is used to pass the swing beam, and can separate a scanning end beam returned from the test specimen from an illumination beam into different light paths. The second lens is set relative to the splitter, and is used to focus the swing beam at the test surface or under the test surface to carry out scanning after forming the scanning end beam.

To achieve the above objective and other objectives, another aspect of the present disclosure provides an optical scanning system, which is applied to scan a test specimen. The optical scanning system includes a system host, the hand-held scanning probe, and a connection cable connecting between the system host and the hand-held scanning probe. The system host therein is provided with a spectrum analyzer and a light source module for providing an optical scanning system light source.

To achieve the above objective and other objectives, still another aspect of the present disclosure provides a hand-held scanning probe, which is included in an optical scanning system to scan a test surface. The hand-held scanning probe includes a housing and an optical component, a fiber-coupled splitter and an interferometer reference end disposed in the housing. The fiber-coupled splitter is used to spilt an optical scanning system light source into a reference end beam and a scanning end beam, and receive the optical scanning system light source. The reference end beam enters the interferometer reference end and is reflected back to the fiber-coupled splitter from the interferometer reference end. The scanning end beam is scattered or reflected through the test specimen corresponding to the test surface, and then also returns to the fiber-coupled splitter.

In an embodiment, the hand-held scanning probe further includes a two-dimensional camera, a third lens and an illumination module disposed in the housing. The two-dimensional camera is set relative to the splitter, not parallel to the direction of the swing beam. The third lens is located between the two-dimensional camera and the splitter. The second lens is located between the splitter and the illuminati on module. The illumination module is for illuminating the test surface.

In an embodiment, the hand-held scanning probe further includes a focus depth adjustment part exposed to the housing. The focus depth adjustment part is for adjusting a distance between the second lens and the test surface of the test specimen.

In an embodiment, the connection cable includes an optical fiber. The fiber-coupled splitter receives the optical scanning system light source through the optical fiber, in order to split the optical scanning system light source into the reference end beam and the scanning end beam. After the reference end beam reflected by the interferometer reference end and the scanning end beam scattered by the test specimen return to the fiber-coupled splitter, they can return to the spectrum analyzer of the system host through the optical fiber to be analyzed to obtain a scanned image.

Accordingly, regarding the hand-held scanning probe and the optical scanning system of the embodiment of the present disclosure, because the first lens and the two-dimensional beam scanning mechanism is provided with a reflector therebetween, an angle of incidence of the laser beam from the first lens may not be perpendicular to an angle of emergence thereof from the second lens, the housing may therefore not have a significantly crooked appearance, thereby reducing the volume, reducing the weight and facilitating the grip by the hand of a person.

### BRIEF DESCRIPTION OF THE DRAWINGS

FIG. 1 is a schematic element configuration of a hand-held scanning probe of a specific embodiment of the present disclosure.

FIG. 2 is a schematic element configuration of an optical scanning system of a specific embodiment of the present disclosure.

FIG. 3 is a schematic solid perspective view of an optical scanning system of a specific embodiment of the present disclosure.

### DETAILED DESCRIPTION OF THE INVENTION

To facilitate understanding of the above purpose, characteristics and effects of this present disclosure, embodiments together with the attached drawings for the detailed description of the present disclosure are provided as below.

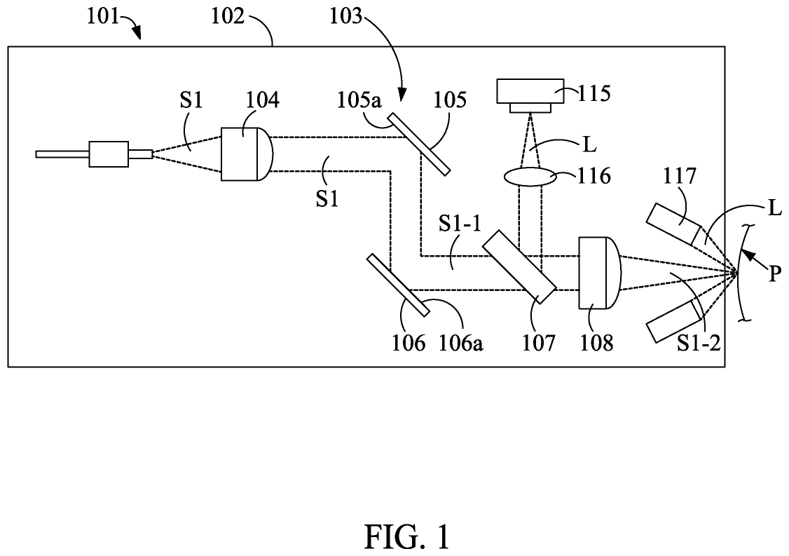

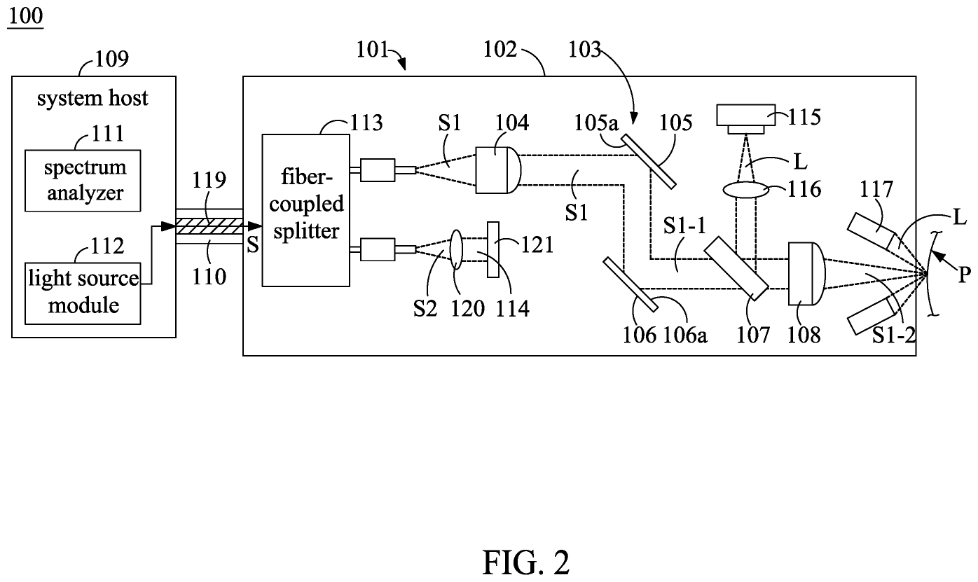

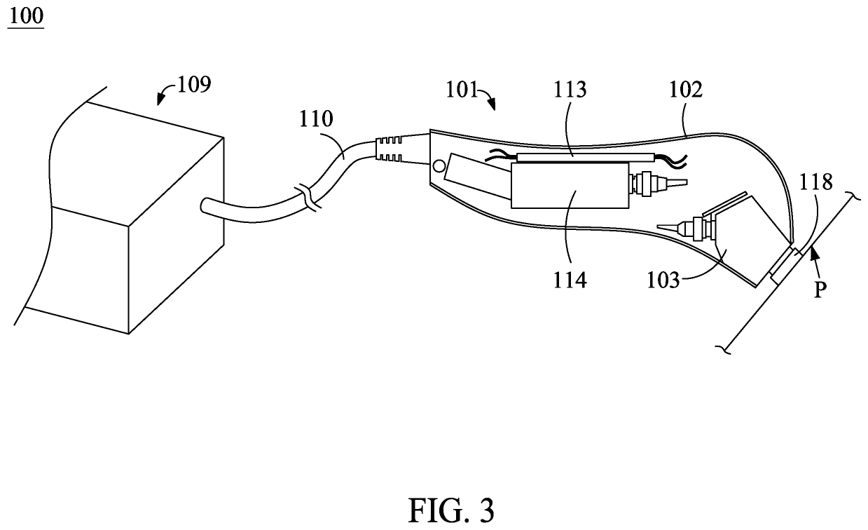

Referring to FIGS. 1 to 3, as shown in FIG. 1, an aspect of the present disclosure provides a hand-held scanning probe 101, which is included in an optical scanning system 100 of FIGS. 2 and 3. The hand-held scanning probe 101 includes a housing 102 and an optical component 103 disposed in the housing 102. The optical component 103 includes a first lens 104, a reflector 105, a two-dimensional beam scanning mechanism 106, a splitter 107 and a second lens 108. The first lens 104 is used to convert a laser beam S1 that is split to a test specimen by a fiber-coupled splitter 113 (shown in FIG. 2) into a form of collimated light. The reflector 105 is set relative to the first lens 104 and has a first mirror surface 105a. The first mirror surface 105a is used to refract the laser beam S1 to change the direction of the laser beam S1. The two-dimensional beam scanning mechanism 106 has a second mirror surface 106a. The second mirror surface 106a is set relative to the first mirror 105a to refract the laser beam S1 again and provide a swing beam S1-1 to a test surface P of the test specimen for two-dimensional mobile scanning, so that it is directed to the splitter 107. The splitter 107 is set relative to the two-dimensional beam scanning mechanism 106, and is used to pass the swing beam S1-1, and can separate a scanning end beam S1-2 returned from the test specimen from an illumination beam into different light paths. The second lens 108 is set relative to the splitter 107, and is used to form the scanning end beam S1-2 after focusing the swing beam S1-1, and carry out scanning at the test surface P or under the test surface P.

As shown in FIGS. 2 and 3, another aspect of the present disclosure provides an optical scanning system 100, which is applied to scan a test specimen corresponding to a test surface P. The optical scanning system 100 includes a system host 109, the hand-held scanning probe 101, and a connection cable 110 connecting between the system host 109 and the hand-held scanning probe 101. The system host 109 therein is provided with a spectrum analyzer 111 and a light source module 112 for providing an optical scanning system light source S. The optical scanning system light source S provided by the light source module 112 may be a near-infrared laser, the wavelength may be 840 nanometers, but is not limited thereto.

As shown in FIGS. 2 and 3, still another aspect of the present disclosure provides a hand-held scanning probe 101, which is included in an optical scanning system 100 to scan a test specimen. The hand-held scanning probe 101 includes a housing 102 and an optical component 103, a fiber-coupled splitter 113 and an interferometer reference end 114 disposed in the housing 102. The fiber-coupled splitter 113 is used to receive an optical scanning system light source S to spilt the optical scanning system light source S into a reference end beam S2 and a laser beam 51. The reference end beam S2 enters the interferometer reference end 114 and is reflected back to the fiber-coupled splitter 113 from the interferometer reference end 114. A scanning end beam S1-2 is scattered through the test surface P and then also returns to the fiber-coupled splitter 113. The interferometer reference end 114 may include a reference end lens 120 and a reference end reflector 121.

As described above, regarding the hand-held scanning probe 101 and the optical scanning system 100 of the embodiment of the present disclosure, because the first lens 104 and the two-dimensional beam scanning mechanism 106 is provided with a reflector 105 therebetween, an angle of incidence of the laser beam S1 from the first lens 104 may not be perpendicular to an angle of emergence thereof from the second lens 108, the housing 102 may therefore not have a significantly crooked appearance, thereby reducing the volume, reducing the weight and facilitating the grip by the hand of a person. As shown in FIG. 3, the housing 102 as a whole has an easy-to-grip streamlined shape, and when gripped by the hand of a person, the hand-held scanning probe 101 can be easily moved on the test surface P without excessive flexion of the wrist.

As shown in FIGS. 1 and 2, the two-dimensional beam scanning mechanism 106 may carry out two-dimensional mobile scanning to the test surface P. Specifically, the swing beam S1-1 may pass through the splitter 107 and the second lens 108 to reach the test surface P and under the test surface P, the scanning end beam S1-2 that is scattered or reflected by the test specimen corresponding to the test surface P may return to the two-dimensional beam scanning mechanism 106 through the second lens 108 and the splitter 107, and then return to the fiber-coupled splitter 113. The splitter 107 may enable near-infrared light to transmit but reflect visible light.

As shown in FIG. 2, in one embodiment, the connection cable 110 includes an optical fiber 119 and a wire (not shown). The fiber-coupled splitter 113 is used to receive the optical scanning system light source S through the optical fiber 119, in order to split the optical scanning system light source S into the reference end beam S2 and the scanning end beam S1-2 of the sample end. After the reference end beam S2 reflected by the interferometer reference end 114 and the scanning end beam S1-2 scattered or reflected by the test specimen return to the fiber-coupled splitter 113, they can return to the spectrum analyzer 111 of the system host 109 through the optical fiber 119 to be analyzed to obtain a scanned image, i.e., an OCT image. The system host 109 is also provided with an instrument control circuit (not shown) or other necessary components. When an optical path difference between the scanning end beam S1-2 and the reference end beam S2 is less than a homology length of the optical scanning system light source S, an interference signal will be generated, which is observed and recorded by the spectrum analyzer 111, and then a depth structure information of the test specimen can be obtained by signal conversion. For example, when the test surface P is the skin surface, microstructures and microvascular images at a depth of about two millimeters under the skin can be reconstructed. The traditional technology usually sets the fiber-coupled splitter 113 and the interferometer reference end 114 in the system host 109, the scanning end beam S1-2 returns a light signal to the system host 109 through the connection cable 110, when the connection cable 110 bends, the scanning end beam S1-2 will produce a change of polarization state and reduce the interference signal and image quality. In the embodiment of the present disclosure, the fiber-coupled splitter 113 and the interferometer reference end 114 are provided in the housing 102 of the hand-held scanning probe 101, when the hand-held scanning probe 101 is moved, the optical fiber 119 for transmitting the reference end beam S2 and for transmitting the scanning end beam S1-2 will also move synchronously, even if the change of polarization state is produced, the polarization change is the same or similar, thereby greatly reducing the difference of two polarization amounts and affecting the signal quality.

As shown in FIGS. 1 and 2, in one embodiment, the hand-held scanning probe 101 may further include a two-dimensional camera 115, a third lens 116 and an illumination module 117 disposed in the housing 102. The two-dimensional camera 115 is set relative to the splitter 107, not parallel to the direction of the swing beam S1-1 (e.g., vertically). The third lens 116 is located between the two-dimensional camera 115 and the splitter 107. The second lens 108 is located between the splitter 107 and the illumination module 117. The illumination module 117 may be a light emitting diode luminary emitting white light for illuminating the test surface P. A distance between the third lens 116 and the second lens 108 may be a sum of a focal length of the two lenses. With reference to FIG. 3, the hand-held scanning probe 101 may further include a focus depth adjustment part 118 exposed to the housing 102. The focus depth adjustment part 118, for example, is a knob for adjusting the focal lengths of the second lens 108 and the third lens 116. Since the test surface P is not necessarily a flat surface, for example, it may be skin and soft and elastic, a distance between the second lens 108 and the test surface may be adjusted by the focus depth adjustment part 118, it is adjusted to an optimal distance and then placed on the test surface P.

As shown in FIGS. 1 and 2, the illumination module 117 may provide an illumination beam L such as white light to the test surface P, the illumination beam

L reflected by the test surface P may be focused on the two-dimensional camera 115 after passing through the second lens 108, the splitter 107 and the third lens 116, so that the two-dimensional camera 115 can obtain a magnified image of the test surface P, the magnification is a focal length ratio of the third lens 116 and the second lens 108. When applied to skin complexion detection of dermatology, the image obtained by the two-dimensional camera 115 may be converted into a signal, and transmitted back to the system host 109, and then reproduced after the signal is restored, so that the inspector can see the magnified image of the skin, in order to replace the function of traditional magnifier, and can assist in the interpretation of OCT images.

While the present disclosure has been described by means of preferable embodiments, those skilled in the art should understand the above description is merely embodiments of the disclosure, and it should not be considered to limit the scope of the disclosure. It should be noted that all changes and substitutions which come within the meaning and range of equivalency of the embodiments are intended to be embraced in the scope of the disclosure. Therefore, the scope of the disclosure is defined by the claims.

What is claimed is:

1. A hand-held scanning probe, included in an optical scanning system, the hand-held scanning probe comprising:
a housing; and
an optical component, disposed in the housing, and including:
a first lens, used to receive a laser beam that is split by a fiber-coupled splitter and convert the laser beam into a form of collimated light;
a reflector, set relative to the first lens and having a first mirror surface, the first mirror surface is used to receive the laser beam to change a direction of the laser beam;
a two-dimensional beam scanning mechanism, having a second mirror surface, the second mirror surface is set relative to the first mirror surface to change the direction of the laser beam again and provide a swing beam to a test surface of a test specimen for two-dimensional mobile scanning;
a splitter, set relative to the two-dimensional beam scanning mechanism, and is used to pass the swing beam, and can separate a scanning end beam returned from the test specimen from an illumination beam into two different light paths; and
a second lens, set relative to the splitter, and is used to focus the swing beam at the test surface or under the test surface to carry out scanning.

2. The hand-held scanning probe according to claim 1, further comprising a two-dimensional camera, a third lens and an illumination module disposed in the housing, the two-dimensional camera is set relative to the splitter, not parallel to the direction of the swing beam, the third lens is located between the two-dimensional camera and the splitter, the second lens is located between the splitter and the illumination module, the illumination module is for illuminating the test surface.

3. The hand-held scanning probe according to claim 2, wherein a distance between the third lens and the second lens is a sum of a focal length of the two lenses, and the hand-held scanning probe further comprises a focus depth adjustment part exposed to the housing, the focus depth adjustment part is for adjusting a distance between the second lens and the test surface.

4. The hand-held scanning probe according to claim 1, further comprising a fiber-coupled splitter and an interferometer reference end disposed in the housing, the fiber-coupled splitter is used to receive an optical scanning system light source to split the optical scanning system light source into a reference end beam and the scanning end beam, the reference end beam enters the interferometer reference end, and the reference end beam is reflected back to the fiber-coupled splitter from the interferometer reference end, the scanning end beam is scattered or reflected through the test specimen, and then also returns to the fiber-coupled splitter.

5. The hand-held scanning probe according to claim 4, wherein the optical scanning system includes a system host, the system host therein is provided with a spectrum analyzer and a light source module for providing the optical scanning system light source, the optical scanning system light source is provided for the fiber-coupled splitter through an optical fiber, after the reference end beam reflected by the interferometer reference end and the scanning end beam scattered or reflected by the test specimen return to the fiber-coupled splitter, they can return to the spectrum analyzer of the system host through the optical fiber to be analyzed to obtain a scanned image.

6. An optical scanning system, applied to scan a test surface, the optical scanning system including a system host, a hand-held scanning probe, and a connection cable connecting between the system host and the hand-held scanning probe,
wherein the system host therein is provided with a spectrum analyzer and a light source module for providing an optical scanning system light source;
wherein the hand-held scanning probe comprises a housing and an optical component disposed in the housing, the optical component including:
a first lens, used to receive a laser beam and convert the laser beam into a form of collimated light, the laser beam is produced by splitting the optical scanning system light source through a fiber-coupled splitter;
a reflector, set relative to the first lens and having a first mirror surface, the first mirror surface is used to receive the laser beam to change a direction of the laser beam;
a two-dimensional beam scanning mechanism, having a second mirror surface, the second mirror surface is set relative to the first mirror surface to change the direction of the laser beam again and form a swing beam to carry out two-dimensional mobile scanning to a test surface of a test specimen;
a splitter, set relative to the two-dimensional beam scanning mechanism, and is used to pass the swing beam, and can separate a scanning end beam returned from the test specimen from an illumination beam into two different light paths; and
a second lens, set relative to the splitter, and is used to focus the swing beam at the test surface or under the test surface to carry out scanning.

7. The optical scanning system according to claim 6, wherein the hand-held scanning probe further comprises a two-dimensional camera, a third lens and an illumination module disposed in the housing and a focus depth adjustment part, the two-dimensional camera is set relative to the splitter, not parallel to the direction of the swing beam, the third lens is located between the camera and the splitter, the second lens is located between the splitter and the illumination module, the illumination module is for illuminating the test surface, a distance between the third lens and the second lens is a sum of a focal length of the two lenses, the focus depth adjustment part is exposed to the housing, and is for adjusting a distance between the second lens and the test surface.

8. The optical scanning system according to claim 6, wherein the hand-held scanning probe further comprises a fiber-coupled splitter and an interferometer reference end disposed in the housing, the connection cable includes an optical fiber, the fiber-coupled splitter receives the optical scanning system light source through the optical fiber, in order to split the optical scanning system light source into a reference end beam and the scanning end beam, the reference end beam enters the interferometer reference end, the reference end beam is reflected back to the fiber-coupled splitter from the interferometer reference end, the scanning end beam is scattered or reflected through the test specimen, and then also returns to the fiber-coupled splitter, after the reference end beam reflected by the interferometer reference end and the scanning end beam scattered or reflected by the test specimen return to the fiber-coupled splitter, they can return to the spectrum analyzer of the system host through the optical fiber to be analyzed to obtain a scanned image.

9. A hand-held scanning probe, included in an optical scanning system to scan a test surface of a test specimen, the hand-held scanning probe comprises a housing, an optical component disposed in the housing, a fiber-coupled splitter disposed in the housing, and an interferometer reference end disposed in the housing,
wherein the fiber-coupled splitter is used to receive an optical scanning system light source to spilt the optical scanning system light source into a reference end beam and a scanning end beam, the reference end beam enters the interferometer reference end, and the reference end beam is reflected back to the fiber-coupled splitter from the interferometer reference end, the scanning end beam is scattered or reflected through the test specimen and then also returns to the fiber-coupled splitter.

10. The hand-held scanning probe according to claim 9, wherein the optical scanning system includes a system host, the system host therein is provided with a spectrum analyzer and a light source module for providing the optical scanning system light source, the optical scanning system light source is provided for the fiber-coupled splitter through an optical fiber, after the reference end beam reflected by the interferometer reference end and the scanning end beam scattered or reflected by the test specimen return to the fiber-coupled splitter, they can return to the spectrum analyzer of the system host through the optical fiber to be analyzed to obtain a scanned image.
